(19) United States
(12) Patent Application Publication
(10) Pub. No.: US 2023/0255627 A1
(43) Pub. Date: Aug. 17, 2023

(54) CURVED SURGICAL STAPLING DEVICE

(71) Applicant: Covidien LP, Mansfield, MA (US)

(72) Inventor: Syed Sarfraz Ahamed, Shanghai (CN)

(21) Appl. No.: 18/009,914

(22) PCT Filed: Jun. 12, 2020
(86) PCT No.: PCT/CN2020/009583

§ 371 Date: Dec. 12, 2022

(51) Int. Cl. A61B 17/072

(52) U.S. Cl. CPC A61B 17/072; A61B 2017/07221; A61B 2017/07257; A61B 2017/07271; A61B 2017/07285

(57) ABSTRACT

A surgical stapling device includes a frame, a jaw that is coupled to the frame, an end effector including an anvil assembly and a cartridge assembly, and a clamp slide assembly. The jaw includes a transverse portion that supports the anvil assembly and a longitudinal portion that includes a guide member that is received within in the cartridge assembly when the cartridge assembly is supported on the clamp slide assembly. The guide member includes guide surfaces that engages a knife blade of the cartridge assembly and a body of the cartridge assembly to support and guide the cartridge assembly as the cartridge assembly moves between spaced and clamped positions to maintain alignment between the cartridge assembly and the anvil assembly.

### FIELD

The present technology is generally related to surgical stapling devices and, more particularly, to surgical stapling devices with a curved end effector.

### BACKGROUND

Surgical stapling devices for applying rows of staples through compressed living tissue are known in the art and are commonly used for closure of tissue or organs prior to transection or resection of the tissue or organs and for occlusion of organs during thoracic or abdominal procedures. Typically, stapling devices include an end effector that may have a variety of configurations including linear, curved, and circular. The end effector may also have a variety of different orientations with respect to a longitudinal axis of the stapling device including aligned or transverse.

In transverse type stapling devices that have curved end effectors, the end effector includes an anvil assembly that is secured to a frame of the stapling device and a cartridge assembly that is supported on a clamp slide assembly of the stapling device. The clamp slide assembly is movable within the frame of the stapling device to move the cartridge assembly towards the anvil assembly along the frame from an open position to a clamped position.

### SUMMARY

Aspects of this disclosure generally relate to a surgical stapling device a frame assembly, a jaw, a clamp slide assembly, an anvil assembly, and a cartridge assembly. The frame assembly defines a channel and includes a proximal portion and a distal portion. The distal portion includes a transverse portion and a longitudinal portion. The jaw includes a transverse portion and a longitudinal portion. The longitudinal portion of the jaw is secured within the channel in the longitudinal portion of the frame assembly. The transverse portion of the jaw includes a guide member that is positioned within and extends from the channel of the frame assembly. The clamp slide assembly is positioned within the channel of the frame assembly and is movable between retracted and advanced positions. The clamp slide assembly includes a distal portion defining a support. The anvil assembly is supported on the jaw and includes a staple forming surface. The cartridge assembly is supported in the support of the clamp slide assembly and includes a body and a knife blade. The cartridge assembly is movable with the clamp slide from a first position spaced from the anvil assembly to a second position in juxtaposed alignment with the anvil assembly. The guide member is received within the body of the cartridge assembly and includes first guide surfaces that engage the cartridge body and a second guide surface that engages the knife blade to guide movement of the cartridge assembly between the spaced and clamped positions.

In aspects of the disclosure, the guide member has a stepped configuration and includes steps on which the first guide surfaces are formed.

In some aspects of the disclosure, the second guide surface includes a horizontal surface and a vertical surface.

In certain aspects of the disclosure, the frame assembly includes a first frame member and a second frame member that are secured together in spaced relation by rivets to define the channel.

In aspects of the disclosure, the distal portion of the frame assembly is defined by a distal portion of each of the frame members, and each of the distal portions of the frame members includes a longitudinal portion and a transverse portion.

In some aspects of the disclosure, the jaw is secured between the longitudinal portions of the distal portions of the frame members.

In certain aspects of the disclosure, each of the frame members is formed of sheet metal and the jaw is formed from stainless steel.

In aspects of the disclosure, the distal portions of the frame members are welded together.

In aspects of the disclosure, the jaw, the anvil assembly, and the cartridge assembly are curved.

In aspects of the disclosure, the stapling device includes a lockout mechanism that is supported in the distal portion of the frame assembly and is operable to prevent movement of the cartridge assembly from the spaced position to the clamped position after the cartridge assembly has been fired.

Other aspects of the disclosure are directed to a stapling device including a frame assembly, a jaw, a clamp slide assembly, an anvil assembly, and a cartridge assembly. The frame assembly includes first and second frame members formed from sheet metal. The first and second frame members are secured to each other to define a channel. Each of the first and second frame members includes a proximal portion and a distal portion. Each of the distal portions include a transverse portion and a longitudinal portion. The jaw is formed from stainless steel and includes a transverse portion and a longitudinal portion. The longitudinal portion of the jaw is secured within the channel between the longitudinal portions of the first and second frame members. The transverse portion of the jaw includes a guide member that is positioned within and extends from the channel of the frame assembly. The clamp slide assembly is positioned within the channel of the frame assembly and is movable between retracted and advanced positions. The clamp slide assembly includes a distal portion that defines a support. The anvil assembly is supported on the transverse portion of the jaw and includes a staple forming surface. The cartridge assembly is supported in the support of the clamp slide assembly and includes a body and a knife blade. The cartridge assembly is movable with the clamp slide from a first position spaced from the anvil assembly to a second position in juxtaposed alignment with the anvil assembly. The guide member is received within the body of the cartridge assembly and includes first guide surfaces that engage the cartridge body and a second guide surface that engages the knife blade to guide movement of the cartridge assembly between the spaced and clamped positions. The guide member has a stepped configuration and includes steps. The first guide surfaces are positioned on the steps.

Other features of the disclosure will be appreciated from the following description.

### BRIEF DESCRIPTION OF DRAWINGS

Various aspects of the disclosure are described herein below with reference to the drawings, wherein:

FIG. 1 is a side perspective view of the disclosed surgical stapling device including aspects of the disclosure;

FIG. 2 is an enlarged view of the indicated area of detail shown in FIG. 1;

FIG. 3 is a frame assembly and anvil assembly of the surgical stapling device shown in FIG. 1 with parts separated;

FIG. 4 is a side perspective view of a jaw of the frame assembly shown in FIG. 3;

FIG. 5 is a cross-sectional view taken along section line 5-5 of FIG. 2;

FIG. 6 is a side perspective view from a first side of an end effector of the surgical stapling shown in FIG. 1 with the cartridge assembly shown in phantom;

FIG. 7 is a side perspective view from a second side of the end effector of the surgical stapling shown in FIG. 1 with the cartridge assembly shown in phantom;

FIG. 8 is a side perspective of the end effector of the stapling device shown in FIG. 1 illustrating a knife of the cartridge assembly with the positioned within the clamp slide assembly with a portion of the clamp slide assembly removed; and

FIG. 9 is a cross-sectional view taken through a cartridge assembly of the end effector of the stapling device shown in FIG. 1.

### DETAILED DESCRIPTION

The disclosed device will now be described in detail with reference to the drawings in which like reference numerals designate identical or corresponding elements in each of the several views. However, it is to be understood that the aspects of the disclosure are merely exemplary of the disclosure and may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure. In addition, directional terms such as front, rear, upper, lower, top, bottom, distal, proximal, and similar terms are used to assist in understanding the description and are not intended to limit the present disclosure.

In this description, the term “proximal” is used generally to refer to that portion of the device that is closer to a clinician, while the term “distal” is used generally to refer to that portion of the device that is farther from the clinician. In addition, the term “endoscopic” is used generally used to refer to endoscopic, laparoscopic, arthroscopic, and/or any other procedure conducted through small diameter incision or cannula. Further, the term “clinician” is used generally to refer to medical personnel including doctors, nurses, and support personnel.

The disclosed surgical stapling device includes a frame, a jaw that is coupled to the frame, an end effector including an anvil assembly and a cartridge assembly, and a clamp slide assembly. The cartridge assembly is supported on the clamp slide assembly and is movable with the clamp slide assembly from a position spaced from the anvil assembly to a position in juxtaposed alignment with the anvil assembly. The jaw includes a transverse portion that supports the anvil assembly and a longitudinal portion that includes a guide member that is received within in the cartridge assembly when the cartridge assembly is supported on the clamp slide assembly. The guide member includes a guide surface that engages a knife blade of the cartridge assembly to support and guide the knife blade as the cartridge assembly moves between the spaced and clamped positions and when the surgical stapling device is fired. The jaw also defines guide steps that engage a body of the cartridge assembly as the cartridge assembly is moved between the spaced and clamped positions to maintain alignment between the cartridge assembly and the anvil assembly.

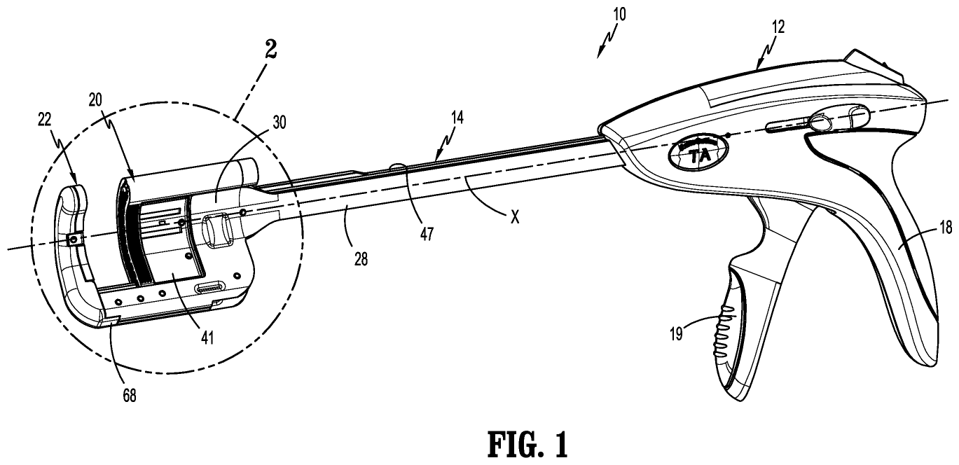

FIG. 1 illustrates the disclosed surgical stapling device shown generally as stapling device 10. The stapling device 10 includes a handle assembly 12, an elongate body 14 that defines a longitudinal axis “X”, and an end effector 16. The handle assembly 12 includes a stationary handle 18 and a trigger 19 that is movable in relation to the stationary handle 18 to actuate the end effector 16, i.e., move the end effector 16 between spaced and clamped positions, and eject staples from the end effector 16. The end effector 16 includes a staple cartridge assembly 20 and an anvil assembly 22 that define axes that are transverse to the longitudinal axis “X” of the elongate body 14. The cartridge assembly 20 includes a body 20a is supported on a distal end portion of the elongate body 14 and is movable in relation to the anvil assembly 22 between the spaced position (FIG. 1) and the clamped position. In the clamped position, the cartridge assembly 20 is in juxtaposed alignment with the anvil assembly 22. In some aspects of the disclosure, the cartridge assembly 20 and the anvil assembly 22 are curved along their transverse axes to facilitate access to certain anatomical regions of a patient during certain surgical procedures, e.g., lower anterior resection surgeries.

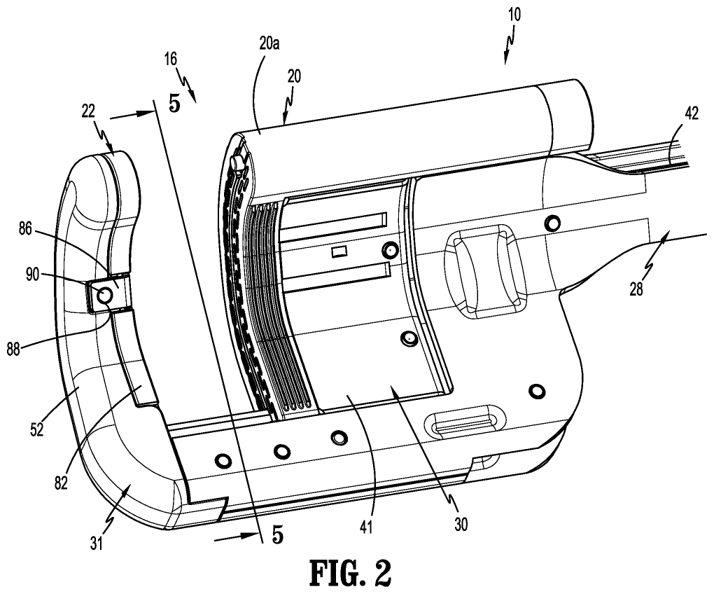

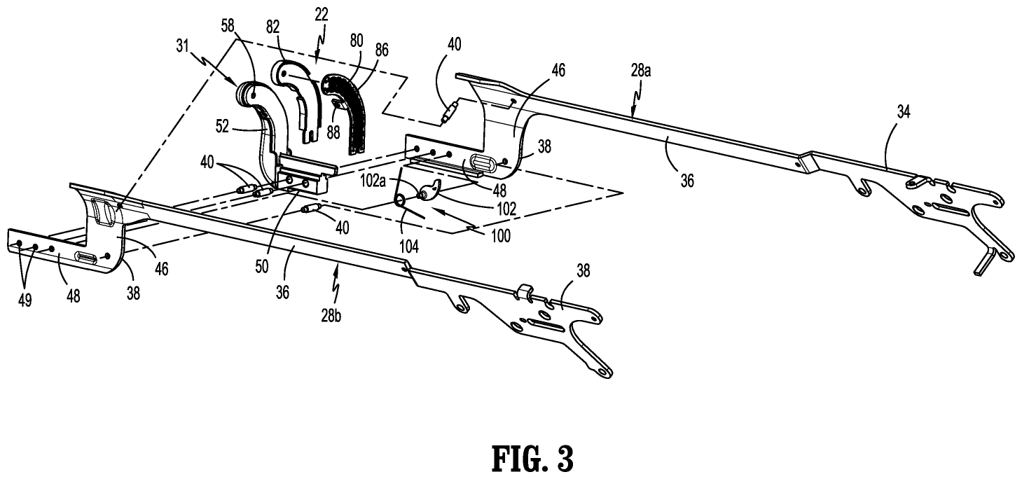

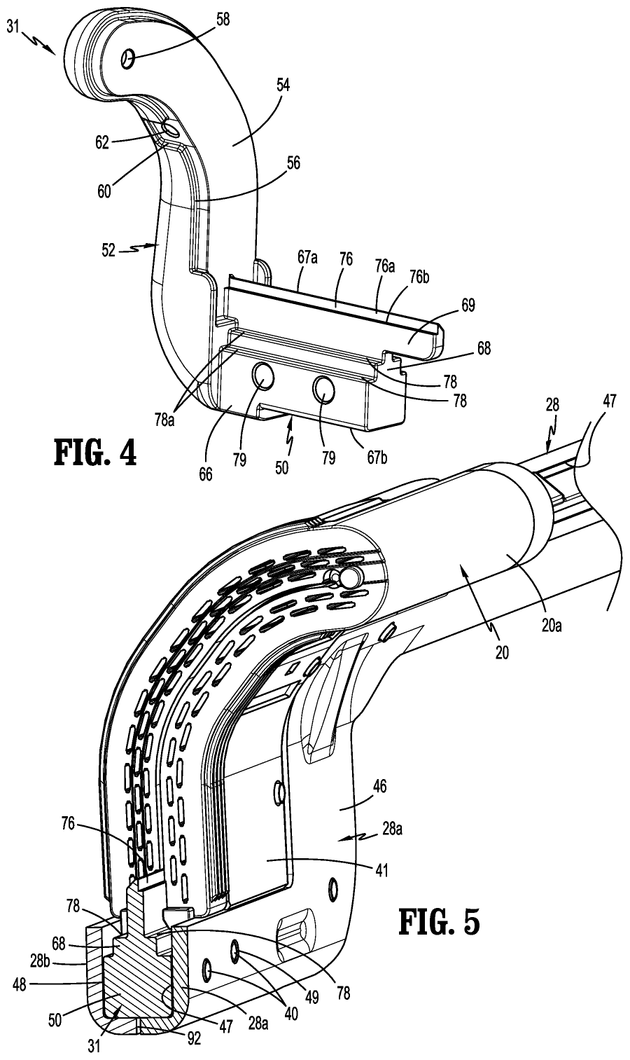

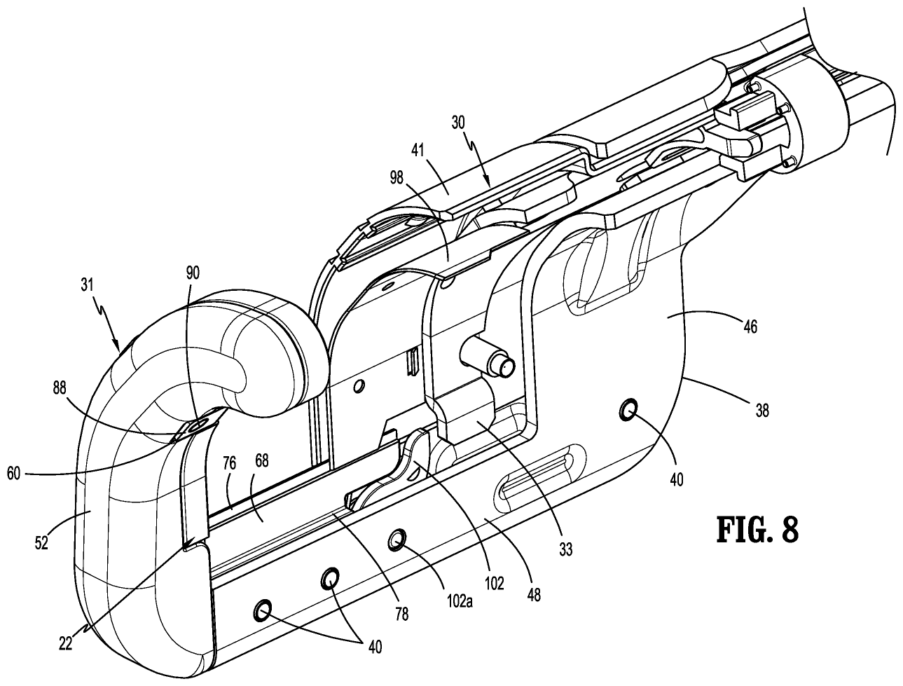

FIGS. 2 and 3 illustrate a frame assembly 28 and a clamp slide assembly 30 of the stapling device 10. The frame assembly 28 defines includes first and second spaced frame members 28a and 28b and a jaw 31. Each of the frame members 28a and 28b (FIG. 3) includes a proximal portion 34 that forms a portion of the handle assembly 12 (FIG. 1), a central portion 36 that forms a portion of the elongate body 14, and a distal portion 38 that forms a portion of the end effector 16 (FIG. 2). The frame members 28a and 28b are secured together with securement members, e.g., rivets 40, in spaced relation to each other to define a frame assembly channel 42 (FIG. 4) between the frame members 28a and 28b. The channel 42 receives the clamp slide assembly 30 and a thrust bar 33 (FIG. 8). The thrust bar 33 is provided to facilitate firing of staples (not shown) from the cartridge assembly 20 into the anvil assembly 22. For a more detailed description of the handle assembly 12 and operation of the thrust bar 33, see U.S. Pat. No. 6,817,508 (“the '508 Patent”). It is envisioned that the frame assembly could be formed of a single component rather than two components as described above.

The clamp slide assembly 30 is received within the channel 42 (FIG. 2) of the frame assembly 28 and includes a distal portion that defines a support 41 for the cartridge assembly 20. The support 40 is moveable in relation to the jaw 31 of the frame assembly 28 from a retracted position to an advanced position to move the cartridge assembly 20 from the spaced position to the clamped position. In aspects of the disclosure, the cartridge assembly 20 is slidably received within the support 40 of the clamp slide assembly 30 and is removable to allow replacement of the cartridge assembly 30 after firing to facilitate reuse of the stapling device 10 (FIG. 1). For a more detailed description of the clamp slide assembly 30 and cartridge assembly 30, see the '508 Patent.

The distal portion 38 of each of the frame members 28a and 28b (FIG. 3) includes a transverse portion 46 and a longitudinal portion 48. The transverse portion 46 is contiguous with the central portion 36 of the respective frame member 28a and 28b and the longitudinal portion 48 extends distally from the transverse portion 46. When the frame members 28a and 28b are coupled together with the rivets 40, the frame members 28a and 28b are spaced from each other to define a channel 47 between the frame members 28a and 28b. Each of the longitudinal portions 48 of the distal portion 38 of each of the frame members 28a and 28b defines through bores 49.

FIG. 4 illustrates the jaw 31 of the frame assembly 28 which includes a longitudinal portion 50 and a transverse portion 52. The transverse portion 52 has a shape that corresponds to the shape of the cartridge assembly 30 and includes a substantially planar proximal surface 54 and side walls 56. In aspects of the disclosure, the transverse portion defines a bore 58 that opens onto the planar surface 54 and the side walls 56 include cutouts 60 and a transverse bore 62. The bore 58 is positioned to receive an alignment pin (not shown) of the cartridge assembly 30 and the cutouts 60 and the bore 62 facilitate securement of the anvil assembly 22 FIG. 3) to the transverse portion 52 as described below.

The longitudinal portion 50 of the jaw 31 includes a body 66 having an inner surface 67a and an outer surface 67b. The body 66 has a stepped configuration and includes a guide member 68 that has a width that decreases in stepped fashion towards the inner surface 67a. The body 66 of the longitudinal portion 50 of the jaw 31 has the smallest width nearer the inner surface 67a of the body 66. The guide member 68 extends proximally beyond the outer surface 67b of the body 66 and defines a knife guide surface 76 that extends longitudinally along a length of the longitudinal portion 50 of the jaw 31. In aspects of the disclosure, the guide surface 76 includes a vertical portion 76a and a horizontal portion 76b as viewed in FIG. 4. The guide member 68 also defines guide steps 78 having guide surfaces 78a (FIG. 4) that extend longitudinally along the length of the longitudinal portion 50 of the jaw 31 and engage and guide the cartridge body 20a of the cartridge assembly 20 as the cartridge assembly 22 is moved between the spaced and clamped positions. The longitudinal portion 50 of the jaw 31 also defines through bores 79.

FIGS. 2 and 3 illustrate the anvil assembly 22 which includes an anvil member 80 and a cutting plate 82. The anvil member 80 includes a proximally facing surface 84 that defines a plurality of staple forming depressions and spaced flanges 86 that define bores 88. The anvil member 80 is secured to the transverse portion 52 of the jaw 31 by rivets or screws 90 (FIG. 2) that extend through the bores 88 in the flanges 86 and into the bores 62 of the jaw 31 (FIG. 4) to secure the flanges 86 within the cutouts 60. The cutting plate 82 is clamped between the transverse portion 52 of the jaw 31 and the anvil member 80 when the anvil member 80 is secured to the transverse portion 52 of the jaw 31.

The longitudinal portion 48 of the distal portion 46 of the frame members 28a and 28b of the frame assembly 28 supports a lockout assembly 100 (FIG. 3). The lockout assembly 100 includes a locking member 102 and a biasing member 104. The locking member 102 is pivotably supported between the frame members 28a and 28b and is movable between a locked position and an unlocked position. The biasing member 104 includes a torsion and is positioned to urge the locking member 102 towards the locked position. The lockout assembly 100 is positioned to prevent readvancement of the cartridge assembly 20 from the spaced position towards the clamped position after the cartridge assembly 20 has been fired. For a detailed description of operation of the lockout mechanism, see the '508 Patent.

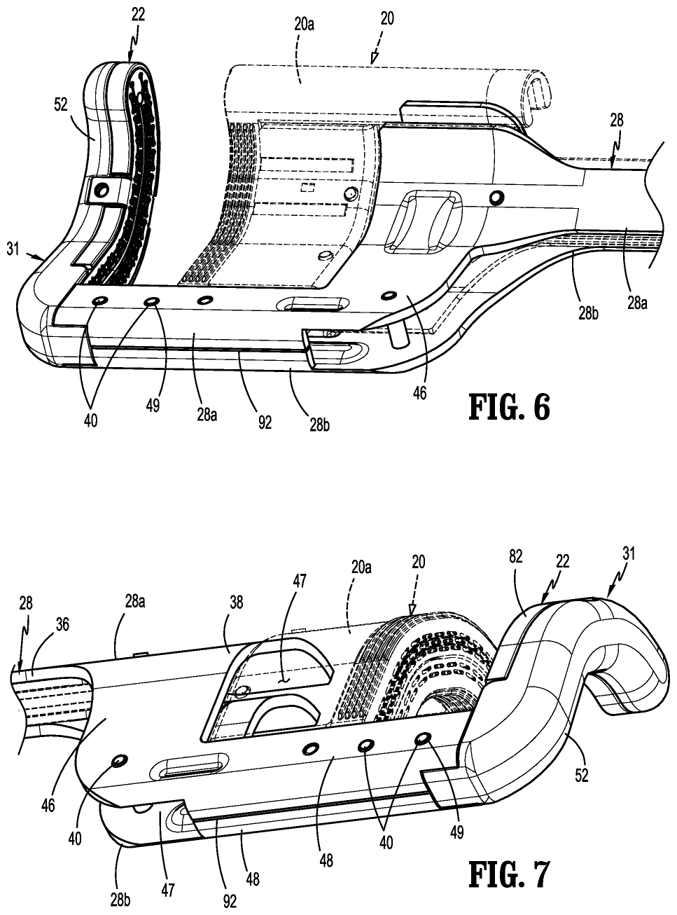

FIGS. 5-7 illustrate the frame assembly 28 and the jaw 31 assembled. The frame members 28a and 28b are secured together and to the jaw 31 by the rivets 40 that extend though the openings 49 in the frame members 28a and 28b and through the through bores 79 (FIG. 4) in the jaw 31. In aspects of the disclosure, the frame members 28a and 28b are formed of sheet metal and the frame members 28a and 28b are also welded together along a weld line 92. In some aspects of the disclosure, the jaw 31 is formed of stainless steel and is clamped between the frame members 28a and 28b.

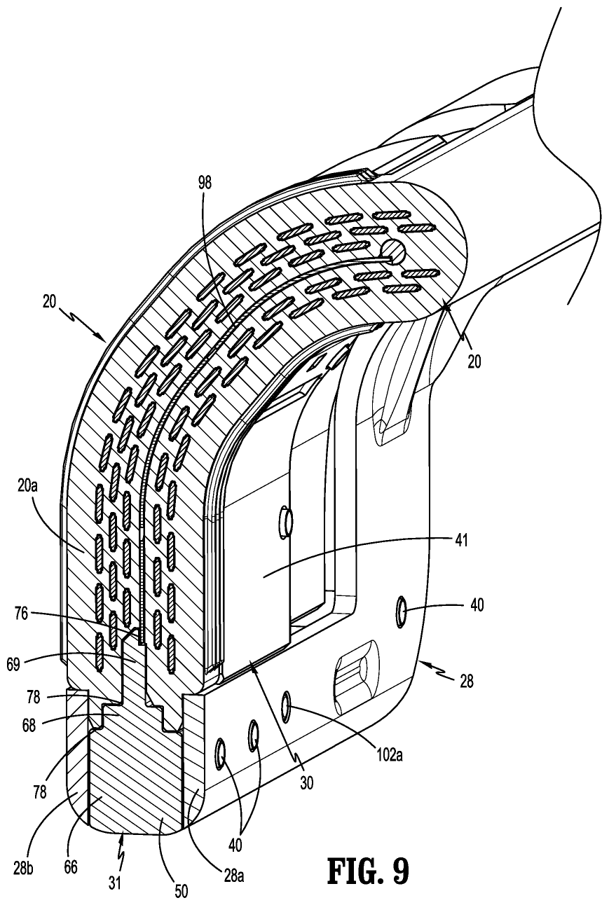

When the jaw 31 is secured between the longitudinal portion 48 of the distal portion 46 of the frame members 28a and 28b, the guide member 68 of the body 66 of the longitudinal portion 50 of the jaw 31 extends inwardly from the channel 47 (FIG. 5) defined between the frame members 28a and 28b such that the guide member 68 projects from the channel 47. When a cartridge assembly 20 is inserted into the support 41 of the clamp slide assembly 30 as illustrated in FIGS. 8 and 9, the guide member 68 of the jaw 31 extends into the cartridge assembly 20 such that the cartridge body 20a engages the guide steps 78 of the guide member 68 of the longitudinal portion 50 of the jaw 31 and a knife blade 98 of the cartridge assembly 20 is supported on the guide surface 76 of the guide member 68. When the clamp slide assembly 30 is moved from its retracted position towards is advanced position to move the cartridge assembly 20 from the spaced position towards the clamped position, the body 20a of the cartridge assembly 20 is guided along the guide steps 78 of the longitudinal portion 50 of the jaw 31 and knife blade 98 moves along the guide surface 76 to maintain the cartridge assembly 20 properly aligned with the anvil assembly 22.

Persons skilled in the art will understand that the devices and methods specifically described herein and illustrated in the accompanying drawings are non-limiting exemplary aspects of the disclosure. It is envisioned that the elements and features illustrated or described in connection with one exemplary embodiment may be combined with the elements and features of another without departing from the scope of the present disclosure. As well, one skilled in the art will appreciate further features and advantages of the disclosure based on the above-described aspects of the disclosure. Accordingly, the disclosure is not to be limited by what has been particularly shown and described, except as indicated by the appended claims.

What is claimed is:

1. A stapling device comprising:
a frame assembly defining a channel and including proximal portion and a distal portion, the distal portion including a transverse portion and a longitudinal portion;
a jaw including a transverse portion and a longitudinal portion, the longitudinal portion of the jaw secured within the channel in the longitudinal portion of the frame assembly, the transverse portion of the jaw including a guide member that is positioned within and extends from the channel of the frame assembly;
a clamp slide assembly positioned within the channel of the frame assembly and movable between retracted and advanced positions, the clamp slide assembly including a distal portion defining a support;
an anvil assembly supported on the jaw, the anvil assembly including a staple forming surface; and
a cartridge assembly supported in the support of the clamp slide assembly, the cartridge assembly including a body and a knife blade, the cartridge assembly movable with the clamp slide from a first position spaced from the anvil assembly to a second position in juxtaposed alignment with the anvil assembly;
wherein the guide member is received within the body of the cartridge assembly and includes first guide surfaces that engage the cartridge body and a second guide surface that engages the knife blade to guide movement of the cartridge assembly between the spaced and clamped positions.

2. The stapling device of claim 1, wherein the guide member has a stepped configuration and includes steps, the first guide surfaces being formed on the steps.

3. The stapling device of claim 2, wherein the second guide surface includes a horizontal surface and a vertical surface.

4. The stapling device of claim 1, wherein the frame assembly includes a first frame member and a second frame member that are secured together in spaced relation by rivets to define the channel.

5. The stapling device of claim 4, wherein the distal portion of the frame assembly is defined by a distal portion of each of the frame members, each of the distal portions of the frame members including a longitudinal portion and a transverse portion, the jaw being secured between the longitudinal portions of the distal portions of the frame members.

6. The stapling device of claim 5, wherein each of the frame members is formed of sheet metal and the jaw is formed from stainless steel.

7. The stapling device of claim 6, wherein the distal portions of the frame members are welded together.

8. The stapling device of claim 1, wherein the jaw, the anvil assembly, and the cartridge assembly are curved.

9. The stapling device of claim 1, further including a lockout mechanism supported in the distal portion of the frame assembly, the lockout mechanism operable to prevent movement of the cartridge assembly from the spaced position to the clamped position after the cartridge assembly has been fired.

10. A stapling device comprising:
a frame assembly including first and second frame members, the first and second frame members formed from sheet metal and secured to each other to define a channel, each of the first and second frame members including a proximal portion and a distal portion, each of the distal portions including a transverse portion and a longitudinal portion;
a jaw formed from stainless steel and including a transverse portion and a longitudinal portion, the longitudinal portion of the jaw secured within the channel between the longitudinal portions of the first and second frame members, the transverse portion of the jaw including a guide member that is positioned within and extends from the channel of the frame assembly;
a clamp slide assembly positioned within the channel of the frame assembly and movable between retracted and advanced positions, the clamp slide assembly including a distal portion defining a support;
an anvil assembly supported on the transverse portion of the jaw, the anvil assembly including a staple forming surface;
a cartridge assembly supported in the support of the clamp slide assembly, the cartridge assembly including a body and a knife blade, the cartridge assembly movable with the clamp slide from a first position spaced from the anvil assembly to a second position in juxtaposed alignment with the anvil assembly;
wherein the guide member is received within the body of the cartridge assembly and includes first guide surfaces that engage the cartridge body and a second guide surface that engages the knife blade to guide movement of the cartridge assembly between the spaced and clamped positions, the guide member having a stepped configuration and including steps, the first guide surfaces being formed on the steps.

11. The stapling device of claim 10, wherein the second guide surface includes a horizontal surface and a vertical surface.

12. The stapling device of claim 10, wherein the first frame member and the second frame member are secured together in spaced relation by rivets to define the channel.

13. The stapling device of claim 10, wherein the distal portions of the first and second frame members are welded together.

14. The stapling device of claim 10, wherein the jaw, the anvil assembly, and the cartridge assembly are curved.

15. The stapling device of claim 10, further including a lockout mechanism supported in the distal portion of the frame assembly, the lockout mechanism operable to prevent movement of the cartridge assembly from the spaced position to the clamped position after the cartridge assembly has been fired.
